(19) United States
(12) Patent Application Publication
(10) Pub. No.: US 2025/0109226 A1
(43) Pub. Date: Apr. 3, 2025

(54) NATIVE CELL MEMBRANE NANOPARTICLES

(71) Applicant: VIRGINIA COMMONWEALTH UNIVERSITY, Richmone, VA (US)

(72) Inventors: Youzhong GUO, Glen Allen, VA (US); Weihua AIU, Glen Allen, VA (US); Thi Kim Hoang TRINH, Ho Chi Minh city (VN)

(21) Appl. No.: 18/581,758

(22) Filed: Feb. 20, 2024

Related U.S. Application Data
(60) Provisional application No. 63/527,594, filed on Jul. 19, 2023.
(60) Provisional application No. 63/527,597, filed on Jul. 19, 2023.
(60) Provisional application No. 63/527,601, filed on Jul. 19, 2023.
(60) Provisional application No. 63/527,598, filed on Jul. 19, 2023.
(60) Provisional application No. 63/527,604, filed on Jul. 19, 2023.

(51) Int. Cl. C08F 228/02; C07K 1/14; C07K 17/08

(52) U.S. Cl. CPC C08F 228/02; C07K 1/145; C07K 17/08

(57) ABSTRACT

Provided is a functionalized polymer useful for extracting, isolating, and/or purifying membrane proteins with their native protein structures being retained. Also provided are a Native Cell Membrane Nanoparticle (NCMN) system which contains the above functionalized polymer and a membrane protein and a method to characterize and/assess the native structure of the membrane protein.

### RELATED APPLICATIONS

This application claims priority from U.S. Provisional Applications Ser. No. 63/527,594 filed on Jul. 19, 2023, Ser. No. 63/527,597 filed on Jul. 19, 2023, Ser. No. 63/527,601 filed on Jul. 19, 2023, Ser. No. 63/527,598 filed on Jul. 19, 2023, and Ser. No. 63/527,604 filed on Jul. 19, 2023, the entire disclosure of which are incorporated herein by this reference.

### GOVERNMENT INTEREST

This invention was made with government support under grant number R01 GM132329 awarded by the National Institutes of Health (NIH). The government may have certain rights in the invention.

### FILED OF THE INVENTION

This invention relates to a Native Cell Membrane Nanoparticle (NCMN) system and methods of preparing and using such system for extracting, isolating and/or solubilizing a membrane protein. In particular, the invention provides a NCMN system which comprises one or more functionalized polymers and a method of using such polymers for extracting the membrane protein of interest while maintaining functional activity and native structure of the isolated membrane protein.

### BACKGROUND

Transmembrane proteins, including channels, transporters, receptors and enzymes, carry out a wide range of vital roles, including controlling what enters and leaves a cell and mediating intracellular communication. Consequently, they are the target of many prescribed drugs, thus there is great interest in understanding the structure and function of this class of proteins. Due to their location within a membrane bilayer and insolubility in water, extracting or solubilizing a membrane protein embedded in a lipid bilayer generally involves buffers and/or reagents containing harsh detergents or surfactants, which destabilize the membrane and interact with the protein, creating a micellar structure around the regions that would normally be in the membrane. However, the use of detergents may present several difficulties, such as regulating efficient extraction concentration of detergent without also denaturing the protein of interest, stripping away annular lipids from the protein which are crucial for function, and/or loss of lateral pressure provided by the membrane which affects both structure and function of the isolated protein. To overcome such limitations, nanodisc is used to stabilize membrane proteins after extracting from cell membrane with a detergent. Another method used in the art involves bicelle, apolipodisc or saposin, which again stabilizes the membrane protein after the detergent-based extraction. However, as apparent from the description, all of the listed strategies of stabilizing the membrane protein still involves the use of detergents in their extraction procedure. For example, U.S. Pat. No. 9,458,191 to Chromy describes a nanolipoprotein particle composition and methods to capture, solubilize and purify membrane proteins. Although the nanolipoprotein composition is able to preserve some structural integrity and activity of the isolated membrane proteins, the disclosed method in Chromy requires a step of mixing the membrane to at least three temperature transition cycles in presence of a detergent to solubilize the target protein. In addition, this method, along with other current methods for membrane protein extraction, notes an additional step of removing the detergent after the isolation of a target protein.

Recently, a styrene-maleic acid (SMA) co-polymer has been used in an alternative method that allows the membrane protein to be extracted without the use of detergents. The SMA co-polymer inserts into a biological membrane and forms small discs of bilayer encircled by the polymer, which are also called lipodiscs or native nanodiscs. The SMA co-polymer forms nanoparticles by absorbing, destabilizing and disrupting the cell membrane by a pH-dependent mechanism. The resulting SMA lipid particle (SMALP)-encapsulated proteins have been shown to be more thermostable. Despite the effective detergent-free membrane protein isolation capability of SMALP, several shortcomings of SMALP have also been apparent: i) being unstable or insoluble in certain environments (e.g., low pH, presence of divalent ions); ii) limited compatibility to only certain groups of membrane proteins (e.g., not compatible for ABC transporters); and iii) involving multiple steps in the preparation stage.

Thus, there is a need in the art for an improved transmembrane protein extraction system with simple and effective isolating properties and ability to maintain the structure and activity of the membrane protein in a detergent-free manner.

### SUMMARY OF THE INVENTION

An object of the invention is a native cell membrane nanoparticle (NCMN) system and a method of using the NCMN system for extracting, isolating and/or solubilizing membrane proteins while preserving native structures and functional activities of the proteins. The NCMN system comprises one or more functionalized polymers. Advantageous features of the present invention include retention of the native membrane protein conformation, an excellent solubility of the NCMN system in aqueous condition and a stability in various environments, especially in the large temperature range (e.g., 0-50° C.) or at low temperature (e.g., 0° C. or 4° C.) or at high temperature (e.g., 40° C.), and/or in a large pH range (e.g., pH=2-11) or at low pH (e.g., pH=4-6) or at high pH=8-10).

The present invention provides novel functionalized polymers for membrane protein research and technology, including effective solubilizing agents and methods for solubilizing, isolating, characterizing membrane proteins, including intrinsic membrane proteins.

In one aspect, the present invention provides functionalized polymers, each of which has: (1) a copolymer backbone and (2) at least one functional branch;
wherein the copolymer or peptide backbone has a number average molecular weight of 1,000-18,000 Da; and
wherein the functional branch is C₁-C₈ straight or branched alkyl substituted with an ionic functional group of

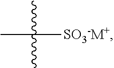

and is attached to an acyl moiety of the copolymer backbone by an amide or ester bonding, or attached to two acyl moieties of the copolymer backbone by an imide bonding;
wherein the alkyl group optionally contains one or more heteroatoms selected from the group consisting of nitrogen, oxygen and sulfur, and M⁺ is NH₄⁺, Na⁺ or K⁺, or M⁺ is another branch of the functionalized polymer which contains a cation selected from the group consisting of

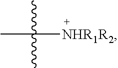

each of R₁ and R₂ independently being H or C₁-C₆ alkyl. In some embodiments, the functional branch is attached to the acyl moiety of the copolymer backbone by an amide or ester bonding and comprises a moiety of the following:

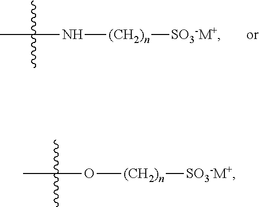

wherein n is 1, 2, 3, 4, 5, or 6; and M⁺ is defined above. In other embodiments, wherein the functional branch is attached to the two acyl moieties of the copolymer or peptide backbone by an imide bonding and comprise a moiety of the following:

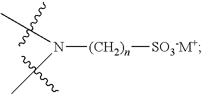

wherein n is 1, 2, 3, 4, 5, or 6; and M⁺ is defined above.

In another aspect, the present invention provides a method of extracting, isolating and/or solubilizing membrane proteins using one or more functionalized polymers of this invention.

The method includes providing a membrane material containing at least one membrane protein and lipid associated to the membrane protein, and contacting the membrane material with one or more functionalized polymers of this invention to form a NCMN system, in which the native structure of the membrane protein is retained.

In further another aspect, the present invention provides a NCMN system, which includes (1) one or more functionalized polymers described above, and (2) a portion of a cell membrane containing at least one membrane protein and membrane lipid associated thereto; wherein the membrane protein and the associated membrane lipid are encapsulated or embedded in one or more functionalized polymers to form a nanoparticle assembly and wherein the native conformation of the membrane protein is retained in the nanoparticle assembly.

In still another aspect, the present invention provides a method of determining/characterizing the native membrane protein structure. The method includes (1) contacting a portion of a cell membrane containing at least a membrane protein and lipid associated thereto with a functionalized polymer of this invention in an aqueous solution to form a nanoparticle assembly, in which the portion of the cell membrane is encapsulated by the functionalized polymer, (2) purifying the nanoparticulate assembly from the aqueous solution, wherein the native structure of the membrane protein is retained in the isolated nanoparticulate assembly, and (3) subjecting the purified nanoparticle assembly to cryo-EM, NMR, or X-ray crystallography to determine and characterize the native structure of the membrane protein.

### DETAILED DESCRIPTION OF THIS INVENTION

In one aspect, this invention features a functionalized polymer that can be used in a native cell membrane nanoparticle (NCMN) system to preserve the native structure of membrane proteins. In this system, the functionalized polymer, together with a membrane protein and its surrounding lipid, are self-assembled into nanodiscs, where the membrane protein and lipid are wrapped, encapsulated, or encased by the functionalized polymer. Such functionalized polymers each have: (1) a copolymer backbone and (2) at least one functional branch;
wherein the copolymer backbone has a number average molecular weight of 1,000-18,000 Da;
wherein the functional branch is C₁-C₈ straight or branched alkyl substituted with an ionic functional group of

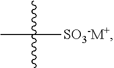

and the functional branch is linked to a carbon atom on the copolymer backbone by an amide or ester bonding, or attached to two carbon atoms on the copolymer backbone by an imide bonding; and
wherein the alkyl group optionally contains one or more heteroatoms selected from the group consisting of nitrogen, oxygen and sulfur, and M⁺ is NH₄⁺, Na⁺ or K⁺, or M⁺ is another branch of the functionalized polymer which contains a cation selected from the group consisting of

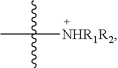

each of R₁ and R₂ independently being H or C₁-C₆ alkyl. The vertical wavy line symbol used in chemical structures herein indicates the bonding site where the chemical moiety shown together with the symbol is bonded with the rest of the molecule.

The term, “polymer” or “polymer derivative”, as used herein, refers to an organic material or polymeric composition consisting of repeated one or more polymerizable units (monomers) joined together, usually in a line, like beads on a string whereas monomers are the basic building blocks of polymers. The term “nanolipoprotein particle”, “nanoparticles”, “nanodisc” or “membrane nanoparticles”, as used herein, indicates a supramolecular complex formed by membrane-forming lipid and a membrane protein and a functionalized polymer, in which the membrane protein and lipid are encased by the polymer. Both scaffold protein and target protein may constitute protein components of the NCMN system. In some embodiments, the membrane-forming lipid constitutes a lipid component of the NCMN system. Preferably, the protein and the lipid are from the same source.

The functionalized polymer of this invention has a backbone derived from a copolymer which contains more than one active acylating groups that can react with a primary or secondary amino group or a hydroxy group. The term “copolymer”, as used herein, refers to a polymer derived from more than one species of monomers. It can be alternating copolymer, random copolymer, or block copolymer. Examples of the copolymer, include but are not limited to, peptide, olefin-acrylic acid copolymer or derivatives thereof, and olefin-maleic acid copolymer and derivatives thereof. The term “derivatives of a copolymer” used herein refers to copolymers wherein the active groups are replaced with their functional or chemical equivalents that lead to the same desired results. For examples, because carboxylic acid groups are equivalent to carboxylic ester and anhydride in reaction with amine to form amide or imide, derivatives of styrene-maleic acid (SMA) copolymer include styrene malate (ester) copolymer and styrene maleic anhydride copolymer. The preferred examples of copolymer are styrene-maleic acid (SMA), styrene-maleic anhydride copolymer (SMAnh), diisobutylene-maleic acid (DIBMA), and diisobutylene-maleic anhydride copolymer (DIBMAnh). The term “acylating group”, as used herein, refers to a functional group containing an acyl (C═O) moiety and ready to react with, and supply the acyl group to, an organic substrate to form a new ester, amide or imide group. Examples of acylating groups include but are not limited to carboxylic acid, di- or poly-carboxylic acid, carboxylate (ester), and carboxylic anhydride.

SMA and DIBMA copolymers can be readily made from radical polymerization of styrene or dibutylene monomers and maleic acid monomers using, e.g., an organic peroxide as the initiator in a various range of molecular weight and maleic content, as evident to those skilled in the art of copolymer synthesis. Likewise, styrene maleic anhydride or diisobutylene maleic anhydride can be made from styrene or dibutylene monomers and maleic anhydride monomers. In addition, SMA, DIBMA, and their maleic anhydride derivative copolymers are also commercially available, for example, SMA® 1000, SMA® 2000, and SMA® 3000 from Total Cray Valley, and Sokalan® CP 9 from BASF.

In certain embodiments, the copolymer has a number average molecular weight between 1,000 Daltons and 30,000 Daltons. The number average molecular weight for such polymers can be determined by several known techniques. A convenient method for such determination is by gel permeation chromatography (GPC) using polystyrene as a calibration reference which additionally provides molecular weight distribution information, see W. W. Yau, J. J. Kirkland and D. D. Bly, “Modern Size Exclusion Liquid Chromatography”, John Wiley and Sons, New York, 1979. In some embodiments, the copolymer has a number average molecular weight between 3,000 Daltons and 25,000 Daltons. In some embodiments, the copolymer has a number average molecular weight between 1,000 Daltons and 18,000 Daltons. In some embodiments, the copolymer has a number average molecular weight between 5,000 Daltons and 18,000 Daltons. In some embodiments, the copolymer has a number average molecular weight between 7,000 Daltons and 16,000 Daltons. In case of SMA or anhydride thereof, the preferred number average molecular weight is between 5,000 and 10,000, or between 6000 and 10,000; more preferably between 6,500 and 8,000 (e.g., about 7,000 or about 7,500). In case of DIBMA and anhydride thereof, the preferred number average molecular weight is between 6,000 and 18,000, more preferably, between 8,000 and 16,000 or between 12,000 and 15,500 (e.g., about 12,000 or about 15,300).

In certain embodiments, the copolymer is an alternating copolymer. In some embodiments, the copolymer is a random copolymer. In some other embodiments, the copolymer is a block copolymer.

In certain embodiments, the copolymer features a ratio of styrene or dibutylene monomer units to maleic acid or anhydride monomer units between 10:1 to 1:10. In some embodiments, the ratio is between 5:1 to 1:5. In some embodiments, the ratio is between 4:1 to 1:1, or between 4:1 and 1.5:1, or between 3:1 and 2:1, or between 2.5:1 to 1.5:1. For example, the copolymer features a ratio of styrene or dibutylene unit to maleic acid or anhydride unit being about 1:1, or about 2:1, or about 3:1 or about 4:1.

It will be understood that, due to the nature of polymerization processes, such monomer ratios are bulk averages, and are not to be taken as descriptive of a particular molecular structure having defined arrangements of monomers. Nevertheless, in general it is to be expected that the monomer types are distributed throughout the copolymer.

It will be also understood that, due to the nature of polymerization reaction, the double bonds in the monomers are transformed into single bonds in order to connect monomers. For example, a maleic acid monomer chemically becomes a succinic acid moiety unit in the polymer, which is referred to herein as either “maleic acid moiety” or “succinic acid moiety” interchangeably.

In the above copolymers, all contain acylating groups, such as monocarboxylic acid retained from an amino acid monomer, or dicarboxylic acid retained from a maleic acid monomer, or anhydride retained from a maleic anhydride monomer. They can react with an amino group or hydroxy group to form an amide, imide, or ester group.

To make the functionalized polymers of this invention, the above copolymers are reacted through condensation reaction with a functional group-containing molecule, which includes (i) at least a primary or secondary amino group or a hydroxy group, and (ii) a sulfonic acid, a sulfonate ester or salt group. Examples of such compounds include, but are not limited to, taurine (2-aminoethanesulfonic acid), 3-aminopropanesulfonic acid, 4-aminobutanesulfonic acid, or sodium, potassium, ammonium salt of the above sulfonic acid, or methyl, ethyl, propyl, or other alkyl ester of the above sulfonic acid. If the sulfonate salt (e.g., NH₄⁺, Na⁺ or K⁺) is used in the reaction, the resulting copolymer product contains at least one functional branch having an ionic group. If sulfonic acid is used, the resulting copolymer is then treated with a basic compound, e.g., a NaOH, KOH, or NH₄OH aqueous solution, and —SO₃H group is then converted to

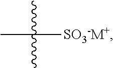

wherein M⁺ is NH₄⁺, Na⁺ or K⁺. If the ester of sulfonic acid is used in the reaction, the resulting copolymer is then hydrolyzed with NaOH, KOH, or NH₄OH aqueous solution, or hydrolyzed with an acid and then neutralized with a base and consequently the sulfonate ester group is converted to

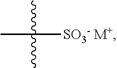

wherein M⁺ is NH₄⁺, Na⁺ or K⁺. Of note, after the hydrolyzation and/or neutralization, the unreacted carboxylic acid/anhydride on the backbone may remain as acid/anhydride or become ionic salt.

In the amidation/imidation/esterification reaction described above, the acylating group (e.g., carboxylic acid or carboxylic anhydride) of the copolymer reacts with the primary or secondary amino group or the hydroxy group of the functional group-containing molecule to form an amide or ester bond. Alternatively, two acylating groups (dicarboxylic acid or carboxylic anhydride) react with a primary amino group of the functional group-containing molecule to form an imide bond. In case of the copolymer containing maleic acid moieties, e.g., SMA or DIBMA, the amino group is reacted with one acid or both of the dicarboxylic acid, to result in one or two branches grafted on the backbone with amide (—C(O)N—) bonding or, if the amino group is primary, it may be reacted with both acids of the maleic acid to result in one branch on the backbone with imide (—C(O)—N—C(O)—) bonding. Similarly, in case of copolymer containing maleic anhydride moieties, e.g., SMAnh or DIBMAnh, one amino group may be reacted with one maleic anhydride moiety to result in open-up of the anhydride ring to provide one branch with amide bonding and one carboxylic acid or two branches with amide bonding, or if the amino group is primary, to result in converting maleic anhydride moiety to a cyclic imide moiety (e.g., succinimide). The structures below illustrate possible reaction products of a maleic acid or anhydride moiety in a copolymer backbone, where L is a linkage organic group (e.g., alkylene) that is associated to both the sulfonic ion and the backbone.

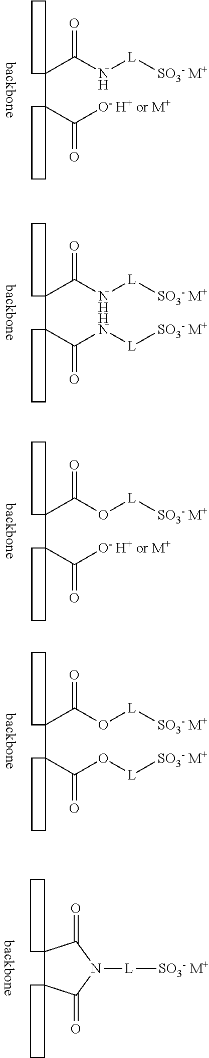

In case that a primary amine compound having a sulfonic or sulfonate group is used, it may first form an amide-grafted functionalized polymer of this invention at a relative low temperature, e.g., room temperature, or below 100° C. When the reaction is elevated to higher temperature, e.g., greater than 100° C., or 150° C., or even 200° C., a more thermostable imide-grafted functionalized polymer of this invention is formed. Both amide or imide grafted functionalized polymer can be readily made. In some embodiments, the functional branch of the functionalized polymer is attached to the acyl moiety of the copolymer backbone by an amide or ester bonding and comprises a moiety of the following:

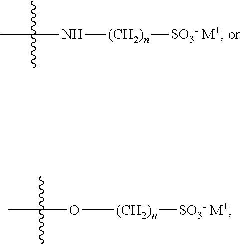

wherein n is 1, 2, 3, 4, 5, or 6; and M⁺ is defined above. In other embodiments, wherein the functional branch is attached to the two acyl moieties of the copolymer or peptide backbone by an imide bonding and comprise a moiety of the following:

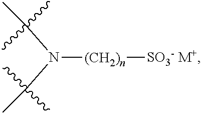

wherein n is 1, 2, 3, 4, 5, or 6; and M⁺ is defined above.

In one embodiment, the functional group-containing molecule has a formula shown below: X—(CH₂)ₙ—SO₃H, wherein X is OH, NH₂, NHR (R being C1-6 alkyl); and n is 1, 2, 3, 4, 5, or 6. An example of the above molecule is 2-aminoethanesulfonic acid (taurine).

Alternatively, one of the above copolymers is reacted with a molecule containing a primary or secondary amino or hydroxy group and an ionic group

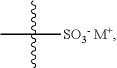

wherein M⁺ is NH₄⁺, Na⁺ or K⁺. Examples of such a molecules are X—(CH₂)ₙ—SO₃⁻Na⁺ or X—(CH₂)ₙ—SO₃⁻K⁺, wherein X is OH, NH₂, NHR (R being C1-6 alkyl); and n is 1, 2, 3, 4, 5, or 6.

The functionalized polymer may further contain, in addition to the above-described functional branch(es), one or more other branches that are different from the functional branch(es) described above. In certain embodiments, such other branch(es) include one or more hydroxy groups. Such other branches may be introduced by reacting a partially grafted functionalized polymer with tris[hydroxymethyl]aminomethane (Tris), 2-amino-2-methyl-1,3-propanediol (AMPDO), 2-amino-1,3-propanediol (APDO), or saccharide or disaccharide or polysaccharide with one hydroxy group being replaced with an amino group, such as d-glucosamine (d-GlcN), d-galactosamine (d-GalN), or d-mannosamine (d-ManN). Alternatively, such other branches may include a cation, e.g.,

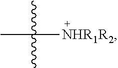

each of R₁ and R₂ independently being H or C₁-C₆ alkyl. A partially grafted functionalized polymer may react with a compound containing a primary or secondary amino group and a tertiary amino group or a compound containing a hydroxy group and a tertiary amino group. Examples of compounds containing a primary or secondary amino group include, but are not limited to, 3-N,N-dimethylethylenediamine (DMEDA), N,N-dimethylpropylenediamine (DMAPA), and 3-(2-(dimethylamino)ethoxy) propylamine (DMAEPA). Examples of compound containing a hydroxy group and a tertiary amino group include, but are not limited to, 2-[2-(dimethylamine)ethyoxy]ethanol, N,N-dimethylamino-ethanol, and 2-dimethylamino-2-2methyl-1-1-propyl. The primary or secondary amino group or hydroxy group of the above compounds react with acylating groups of the copolymers via condensation. Under certain conditions known in the art, the proton of —SO₃H on one branch can transfer to the tertiary amino group on another branch to form a zwitterion in the polymer (aka intramolecular acid-base reaction).

In some embodiments, the functionalized polymer contains the functional branch described above and further contains one or more amide-grafted branches, which each contains two or more hydroxy groups. In other embodiments, the functionalized polymer contains the functional branch described above and further contains one or more imide-grafted branches, which each contains two or more hydroxy groups. In some further embodiments, the functionalized polymer contains the functional branch described above and further contains one or more amide-grafted branches, which each contains

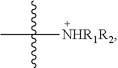

each of R₁ and R₂ independently being H or C₁-C₆ alkyl. In some further embodiments, the functionalized polymer contains the functional branch described above and further contains one or more imide-grafted branches, which each contains

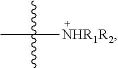

each of R₁ and R₂ independently being H or C₁-C₆alkyl.

The copolymers used to prepare the functionalized polymers of this invention may contain a plurality of acylating groups, e.g., multiple maleic acid/anhydride monomer units. It is understood that one skilled in the art of chemistry can control the reaction conditions to have all or part of the acylating groups react with the functional group-containing molecule, which results in different levels of grafting, for example, by manipulating the molar ratio of copolymer to functional group-containing molecule. The “grafting ratio” or “grafting degree” used herein referred to the percentage of acylating groups that have been transformed by reacting with the functional group-containing molecules to provide functional branches, relative to all acylating groups available prior to the condensation reaction with the functional group-containing molecule. For the purpose of calculation of grafting degree, one maleic anhydride moiety contributes two acyl groups and therefore is considered to have two acylating groups. If one maleic anhydride moiety reacts with a primary amine to form an imide moiety or it reacts with two molecules of amine to form two amide moieties, then both acylating groups in the anhydride have been reacted. If one maleic anhydride moiety reacts with one molecule of amine to form one amide moiety and one acid moiety, then the amide moiety is considered as “reacted” and “grafted” and the acid moiety is considered “not reacted” and “not grafted”. In some embodiments, the functionalized polymer has a grafting ratio of 100%. In some other embodiments, the functionalized polymer has a grafting ratio of 1-75%, or 20-30%, or 25-50%, or 25-75%, or above 15%, or above 25%, or above 35%, or above 50%, or above 75%. In some other embodiments, the functionalized polymer has a grafting ratio of about 15%, about 25%, about 50%, or about 75%, or about 100%.

In some embodiments, the copolymer of styrene (or diisobutylene) and maleic acid/anhydride is labelled to aid in identification. Suitable labels include, for example, fluorescent materials and radioisotopes.

The functionalized polymer is partially or completely soluble in water. A stock solution of the polymer is prepared in distilled water and kept for storage at varying concentrations, e.g., 5% (w/v), or 10% (w/v) at an appropriate temperature.

Examples of the functionalized polymers are:

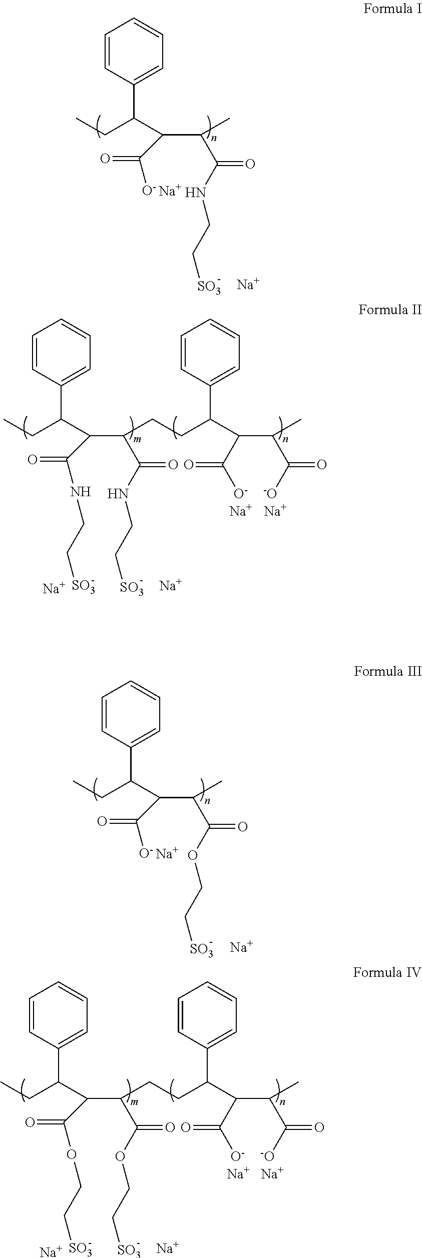

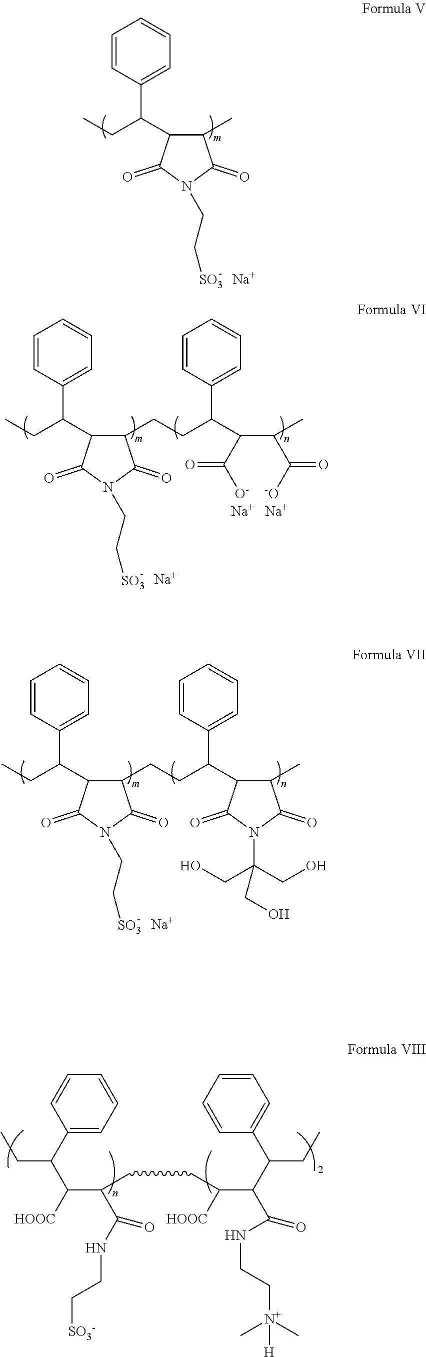

In another aspect, this invention features a NCMN system and/or aNCMN assembly, in which a membrane protein and its associated membrane lipid are encapsulated, encased, or embedded in one or more functionalized polymers described above. The terms, “encapsulated”, “encased”, and “embedded”, as used herein, refer to a state where a portion or entirety of the membrane protein of interest is enclosed, stored or hidden inside a polymer or a system comprising a polymer. The NCMN system may further comprise a suitable medium in which the NCMNs are stably dissolved or dispersed. Examples of such media include but are not limited to water, an aqueous buffer solution, and polar organic solvents like DMSO or DMF.

In preferred embodiments, the NCMN system and NCMN assembly may comprise one or more functionalized polymer-encapsulated membrane proteins in their native structures. The term “Native structure”, “Native form”, “Native environment” or “Native state”, as used herein, refers to a protein's properly folded and assembled form with operative structure and function. It is a biochemical acceptable formation of all four levels of structures including secondary, tertiary, and quaternary structures formed by weak interactions along the covalently-bonded backbone when the proteins are expressed in their designated cellular locations. The proteins in their native states with intact structures that are not altered by heat, chemicals, enzyme reaction or other denaturants are referred to “native proteins”. The “retained native structure” means that the structure of the membrane protein in the NCMN system is substantially identical (e.g., ≥90% identical) to the structure of the protein in its native membrane environment. The retention of the native protein structure can be evidenced by manifestation of the native activity of the membrane protein in the NCMN system.

The term “membrane protein”, “membrane associated protein”, “target protein” or “target membrane protein” as used herein indicates any protein having a structure that is suitable for attachment to or association with a biological membrane or biomembrane (i.e., an enclosing or separating amphipathic layer that acts as a barrier within or around a cell). In particular, target proteins include proteins that contain large regions or structural domains that are hydrophobic (the regions that are embedded in or bound to the membrane); those proteins can be extremely difficult to work with in aqueous systems, since when removed from their normal lipid bilayer environment those proteins tend to aggregate and become insoluble. Accordingly, target proteins are proteins that typically can assume an active form wherein the target protein exhibits one or more functions or activities, and an inactive form wherein the target protein do not exhibit those functions/activities. Exemplary target proteins include, but are not limited to, membrane proteins, i.e., proteins that can be attached to, or associated with the membrane of a cell or an organelle, such as integral membrane proteins (i.e., proteins or assembly of proteins that are permanently attached to the biological membrane), or peripheral membrane proteins (i.e., proteins that adhere only temporarily to the biological membrane with which they are associated). Peripheral membrane proteins are proteins that attach to integral membrane proteins or penetrate the peripheral regions of the lipid bilayer with an attachment that is reversible. Generally, integral membrane proteins may be separated from the biological membranes using detergents, nonpolar solvents, or sometimes denaturing agents. However, in preferred embodiments, the systems and methods of the present invention are substantially free of detergents or any other harsh denaturing agents. As used herein, the term “substantially free” means less than 0.01 wt. %. In other embodiments, the system may be free of a detergent but contain one or more nonpolar solvents.

Some exemplary membrane proteins include, but are not limited to, a G protein-coupled receptor, a 5-hydroxytryptamine receptor, an acetylcholine receptor, an adenosine receptor, an angiotensin receptor, an apelin receptor, a bile acid receptor, a bombesin receptor, a bradykinin receptor, a cannabinoid receptor, a chemerin receptor, a chemokine receptor, a cholecystokinin receptor, a Class A Orphan receptor, a dopamine receptor, an endothelin receptor, a formyl peptide receptor, a free fatty acid receptor, a galanin receptor, a ghrelin receptor, a glycoprotein hormone receptor, a gonadotrophin-releasing hormone receptor, a G protein-coupled estrogen receptor, a histamine receptor, a hydroxycarboxylic acid receptor, a kisspeptin receptor, a leukotriene receptor, a lysophospholipid receptor, a lysophospholipid SIP receptor, a melanin-concentrating hormone receptor, a melanocortin receptor, a melatonin receptor, a motilin receptor, a neuromedin U receptor, a neuropeptide FF/neuropeptide AF receptor, a neuropeptide S receptor, a neuropeptide W/neuropeptide B receptor, a neuropeptide Y receptor, a neurotensin receptor, an opioid receptor, an opsin receptor, an orexin receptor, an oxoglutarate receptor, a P2Y receptor, a platelet-activating factor receptor, a prokineticin receptor, a prolactin-releasing peptide receptor, a prostanoid receptor, a proteinase-activated receptor, a QRFP receptor, a relaxin family peptide receptor, a somatostatin receptor, a succinate receptor, a tachykinin receptor, a thyrotropin-releasing hormone receptor, a trace amine receptor, a urotensin receptor, a vasopressin receptor, U-¹⁵N Cytb5, cytochomromes such as cytochrome b5, cytochrome P450, cytochrome P450 reductase, cytochrome c, or a combination of two or more thereof.

The term, “membrane”, “lipid bilayer”, “membrane forming lipid” or “amphipathic lipid”, as used herein, indicates a lipid possessing both hydrophilic and hydrophobic properties that in an aqueous environment assemble in a lipid bilayer structure that consists of two opposing layers of amphipathic molecules known as polar lipids. Each polar lipid has a hydrophilic moiety (e.g., a polar group), such as a derivatized phosphate or a saccharide group, and a hydrophobic moiety (e.g., a long hydrocarbon chain). Exemplary polar lipids include phospholipids, sphingolipids, glycolipids, ether lipids, sterols and alkyl phosphocholines. Amphipathic lipids include but are not limited to membrane lipids, i.e., amphipathic lipids that are constituents of a biological membrane, such as phospholipids like dimyristoylphosphatidylcholine (DMPC) or Dioleoyl phosphoethanolamine (DOPE) or dioleoyl phosphatidylcholine (DOPC). The membrane forming lipid can assume different states in an aqueous environment, including a frozen gel state and/or a fluid liquid-crystalline state, wherein each state is associated with one or more temperatures or pHs at which the particular structural phase is detectable. The NCMN system may include at least a portion of the “native membrane” (i.e., the membrane surrounding the one or more membrane proteins of interest), which may be in a combined form with the one or more membrane proteins and at least one polymer. The “associated membrane lipid” preferably is a membrane lipid which, in a native cell, is found in the same membrane as the membrane protein. In some embodiments, the associated membrane lipid is a membrane lipid which, in a native cell, surrounds the membrane protein to form a native protein-lipid complex.

The membrane protein can be any protein that interacts with or is part of a biological lipid bilayer membrane, and can be permanently anchored or temporarily anchored to a lipid bilayer membrane. In some embodiments, the membrane protein included in the NCMN assembly or system spans across at least one half of the lipid bilayer, from one hydrophilic face to the center of the hydrophobic edge. In some cases, the membrane protein spans across the entire lipid bilayer from the first hydrophilic face to the second hydrophilic face at least once. In some cases, the membrane protein spans across the entire lipid bilayer from the first hydrophilic face to the second hydrophilic face more than once.

In yet another aspect, this invention provides a method of preparing such a NCMN system or NCMN assembly by contacting the functionalized polymer with a cellular material containing a membrane protein and its associated membrane lipid. The term “contact” refers to the act of touching, making contact, mixing, or bringing to immediate or close proximity, including at the molecular level, for example, to bring about a chemical reaction or physical change, e.g., in a solution or other reaction mixture.

The membrane protein and its associated membrane lipid in the cellular material can be from the same source or different sources. Preferably, they are from the same source.

The cellular material comprising a membrane protein and associated lipid may be whole cells. Yet, it can also be lysed or purified membrane material. Such lysed membrane material may be partly or substantially free of whole cells. Such purified membrane material may be partly, substantially or completely free of other cellular material. For example, providing purified membrane material may comprise centrifugation of lysed cells in a manner known in the art.

It has been found that the presence of DNA in a solution of macromolecular assemblies can increase viscosity and lead to difficulties in purification. Therefore, it may be advantageous to ensure that the macromolecular assemblies produced by the present method are at least substantially free of nucleic acid, either by separating the membrane material from other cellular components before macromolecular assembly, or by treatment of the liquid containing the assemblies.

One skilled person in the art of biochemistry, in particular membrane protein science, can readily choose conditions, e.g., temperature, medium, buffer, pH, and devices, to preparing a NCMN system or NCMN assembly by contacting the functionalized polymer with a cellular material containing a membrane protein and its associated membrane lipid. An exemplary protocol of preparing Native Cell Membrane Nanoparticles (NCMN) containing membrane protein for research analysis is described below:
1. Resuspend 1 g of membrane pellet in 10 mL NCMNs Buffer A of Table 1.
2. Homogenize the resuspended cell membrane sample with a glass Dounce homogenizer at 20° C.
3. Transfer the suspended membrane sample to a 50 mL polypropylene tube and add membrane active polymers stock solution and additional NCMNs Buffer A to bring the sample to a final concentration of 2.5% (w/v) NCMN membrane active polymer. Stock solutions of membrane active polymers is made in double distilled water and can be kept at varying concentrations, but typically 10% (w/v).
4. Shake the sample for 2 h at 20° C.
5. Load the sample into an ultracentrifuge and spin at 150,000×g for 1 h at 20° C.
6. While the sample is being ultra-centrifuged begin to equilibrate a 5 mL Ni-NTA column with 25 mL of NCMNs Buffer A.
7. Collect the supernatant after ultracentrifugation is complete and load it onto 5 mL of Ni-NTA column at room temperature with a flow rate of 0.5 mL/min using a syringe pump.
8. Wash fast protein liquid chromatography (FPLC) lines with enough NCMN Buffer B (Table 1) to completely flush the system and then connect the column to the FPLC machine.
9. Wash the column with 30 mL of NCMN Buffer B with a flow rate of 1 mL/min and collect the flow through.
10. Wash the column with 30 mL of NCMNs Buffer C (Table 1) with a flow rate of 1 mL/min and collect the flow through.
11. Elute the protein with 20 mL of NCMNs Buffer D (Table 1) at a flow rate of 0.5 mL/min and collect the sample using a fraction collector and the fractions each being set to 1.0 mL.
12. Store the protein samples at 4° C.
13. Run an SDS-PAGE gel electrophoresis assay in order to check the samples that correspond to peaks observed on the FPLC elution graph.

TABLE 1
List of NCMN purification buffers

| Buffer A | Buffer B | Buffer C | Buffer D | Buffer E |
|---|---|---|---|---|
| 50 mM | 25 mM | 25 mM | 25 mM | 40 mM |
| HEPES, | HEPES, | HEPES, | HEPES, | HEPES, |
| pH 8.4 | pH 7.8 | pH 7.8 | pH 7.8 | pH 7.8 |
| 500 mM | 500 mM | 500 mM | 500 mM | 200 mM |
| NaCl | NaCl | NaCl | NaCl | NaCl |
| 5% | 5% | 5% | 5% | 0.1 mM |
| glycerol | glycerol | glycerol | glycerol | TCEP |
| 20 mM | 40 mM | 75 mM | 300 mM | |
| Imidazole | Imidazole | Imidazole | Imidazole | |
| 0.1 mM | 0.1 mM | 0.1 mM | 0.1 mM | |
| TCEP | TCEP | TCEP | TCEP | |

The above-described exemplary protocol is for illustrative purposes only and is not meant to be limiting. Alternative or modified protocols and embodiments will be apparent to those of skill in the art in view of this disclosure.

In some embodiments, contacting the functionalized polymer with the cellular material is carried out at a temperature of at least 4° C., or at least 10° C., or at least 15° C. or at least 20° C. Additionally or alternatively, contacting the functionalized polymer with the cellular material may be carried out at a temperature no lower than the gel to liquid phase transition temperature of the cellular material. One of the advantages of this invention is the NCMN system is stable over a large temperature range, e.g., from 4° C. to 40° C. In certain embodiments, the above operation can be carried out at relative high temperature, e.g., from 20° C. to 40° C.

Contacting the functionalized polymer with the cellular material is carried out over a large pH range, e.g., from pH of 2-11. In some embodiments, the contacting step can be carried out at a pH of between 6.5 and 9, between 3 and 7, between 4 and 7, between 5 and 7, between 6 and 7, between 7 and 11, between 7 and 10, between 7 and 9. In some further embodiments, it is carried out at a pH of between 7 and 8.

In some embodiments, the functionalized polymer and the cellular material are independently and separately dissolved or dispersed in aqueous solutions at a suitable concentration prior to the contacting step. The aqueous solution containing the functionalized polymer is then mixed with the aqueous solution containing the cellular material. The resulting NCMN assembly is hereby solubilized or dispersed in the aqueous solution.

It is well known in biochemical research that, when working with proteins (and particularly where it is desired to maintain the proteins in their native conformations), any processing should be carried out at low temperature. Thus, processing of proteins (such as extraction) is commonly performed in a refrigerated room. The purpose of operation at low temperature is to prevent any degradation of the protein, including (but not limited to) denaturation and oxidation. However, at such low temperatures, cellular material has a tendency to form a gel-like structure, with the result that the processing is difficult to achieve and takes longer than desired.

It has surprisingly been found that the method of the invention stabilizes the protein to such an extent that processing can be carried out at higher temperatures. In particular, processing can be carried out at or above the gel to liquid phase transition temperature, thereby reducing the time required to solubilize the protein (in the form of the macromolecular assemblies) without sacrificing the protein stability.

The gel to liquid phase transition temperature is well understood by those in the art, and is known to be affected by the acyl chain lengths in the phospholipids of a phospholipid bilayer. Many lipids in bacteria and humans have chain lengths of between 12 and 14 carbon atoms, giving a transition temperature of between 2° and 40° C.

In some embodiments, mixing the polymer with the cellular material additionally comprises sonication of the mixture. This aids in mixing of the cellular material with the functionalized polymer by breaking up membranes in the cellular material.

In certain embodiments, a functionalized polymer has been labelled by e.g., fluorescent materials and radioisotopes, and, as a result, the resulting NCMN system and assembly is also labelled for identification.

In still another aspect, this invention provides a method of solubilizing one or more membrane proteins or membrane associated proteins in an aqueous medium by contacting a membrane material containing such proteins with one or more functionalized polymers in said aqueous medium to form a NCMN system in which the nanoparticles are soluble or more soluble in said aqueous medium. In further another aspect, this invention provides a method of using the NCMN system to extract, isolate, purify, and/or concentrate one or more membrane proteins for preserving the native lipid environment and/or maintaining the native structure and activity of the target membrane proteins. Each of these extracting/isolating/purifying/concentrating methods comprises steps of: contacting a cell membrane with an effective amount of one or more functionalized polymers; extracting or isolating one or more membrane proteins embedded in their native membrane within the NCMN system. For contacting a cell membrane with at least one functionalized polymer, the cell membrane and the functionalized polymer may be mixed at a ratio of about 5:1 to 1:10, preferably about 4:1 to 1:8, more preferably about 3:1 to 1:5. Such cell membranes are cell membranes in either prokaryotes or eukaryotes, or the membranes surrounding many intracellular organelles in eukaryotes.

The term “contacting” is defined above. An “effective amount” used herein generally means an amount which provides the desired effect. In the present disclosure, an effective amount refers to a concentration or weight of the polymer to entirely or partially extract and/or purify the membrane protein of interest from the cell membrane while maintaining the protein's functional activity and native structure. For example, to extract and/or purify the membrane protein of interest, 1-100 mg, preferably 1-80 mg, or more preferably 1-50 mg of NCMN system may be used. In some embodiments, more than 100 mg of NCMN may be used. In other embodiments, less than 1 mg of NCMN may be used. In addition, an “appropriate time” refers to a time period to obtain the desired effect. For example, in the present disclosure, an appropriate time refers to a time for the functionalized polymers to extract, isolate or purify the membrane protein of interest into a NCMN system. Alternatively, an appropriate time or amount may represent the time or amount that is required for maintaining the isolated membrane protein's structure and functional activity. For example, the isolated membrane protein may be stable in a NCMN system for hours (e.g., any time between 1-24 hours), days (e.g., any day between 1-30 days), months (e.g., any month between 1-12 months) or years (e.g., 1-3 years). The phrase “one or more” is readily understood by one of skill in the art, particularly when read in context of its usage. For example, one or more of functionalized polymers refers to one to three, or one to up to four.

The terms, “extract” and “isolate”, as used herein, are used interchangeably and refer to a series of processes intended to isolate one or more proteins from a complex, such as cells, tissues, whole organisms, or cellular membranes. The process described herein refers to a method that separates one or more target membrane proteins and non-protein parts of the mixture (i.e., a portion of the membrane or a lipid bilayer). In addition, the terms may represent extraction and isolation of a specific protein of interest from all other proteins or non-protein components.

The term, “solubilize” as used herein, indicates to make a membrane protein susceptible or more susceptible to dissolve in a medium and in particular in an aqueous medium. Accordingly, when used with reference to a membrane associated protein the term solubilize indicates making the membrane associated protein soluble or more soluble (susceptible of being dissolved) into an aqueous environment and encompasses solubilizing proteins from a pellet, a solution, a membrane fraction and any other medium and/or preparations wherein the membrane associated protein is comprised alone or in combination with other compounds and/or molecules.

The terms, “purify” and “concentrate” as used herein, indicate the process of freeing the membrane protein from other components. In particular with reference to a membrane associated protein, the term “purify” indicates the act of separating the membrane associated protein from a medium wherein the protein is comprised together with other molecules and encompasses purification of membrane associated proteins from molecular and/or biological structures such as membranes or molecular complexes. Accordingly, “purifying” a membrane associated protein into a nanoparticle indicates the act of separating the membrane associated protein from an original environment and/or cellular location into the nanoparticle particle.

The methods herein described can be used, in several embodiments, to assemble, to extract, to solubilize and/or to purify many kind of membrane proteins or membrane associated proteins of interest, including integral membrane proteins and any other proteins difficult to isolate without applying a detergent. In some embodiments, the NCMN system may comprise at least one NCMN polymer. In other embodiments, the NCMN system may comprise combinations of functionalized polymers. In some cases, the NCMN system comprises one or more functionalized polymers described above together with one or more other active compounds, for example, one ore more NCMN polymers described in US 20230295349A1.

The NCMN system provides an improved tolerance to low pH conditions during the protein extraction. Further, the functionalized polymers can isolate and extract materials in a simplified step by eliminating the use of detergents. As such, detergent titration and optimization for reconstituting the isolated protein, as well as several wash steps for removal of detergent after the reconstitution, are not required, thereby increasing the efficiency and overall yield of NCMN containing liposomes.

The selection process of at least one NCMN polymer in the NCMN system may be based on the pH, temperature, molecular or chemical structure, functional activity or any other factors of a specific membrane protein and/or the lipid bilayer environment of the protein. The NCMN system may be used for membrane proteins in a condition at a low pH (i.e., pH<6, 5, 4, or 3), a neutral pH (i.e., pH=7) and/or a high pH (i.e., pH>8, 9, or 10). In some embodiments, additional pH-buffer modifying solution may be included. One or more selected functionalized polymers in the system may be stable in a certain pH conditions to preserve the native structure of the membrane protein.

In some embodiments, the methods and NCMN system herein disclosed allow functionalized polymers to incorporate diverse membrane proteins, which include, but are not limited to, integral membrane proteins containing transmembrane alpha-helices and/or beta-sheet structures, as well as, peripheral and monotopic membrane proteins, G protein receptors, Type I, II and III cell-surface receptors and the likes. Membrane proteins that have single or multiple membrane spans can also be functionally solubilized into a NCMN system. Other suitable applications of the NCMN system include various biological fields, such as for detecting microorganisms and/or analysis of bacterial or viral and host protein-protein interactions. The methods of use may also include drug delivery by serving as vehicles for therapeutic based countermeasures.

In further another aspect, this invention provides a method of characterizing a membrane protein or determining the structure of a membrane protein. The method includes providing a NCMN system described above, which contains a nanoparticle assembly of a functionalized polymer, a membrane protein with the native structure, and lipid, and characterizing, or determine the structure of, the membrane protein in the nanoparticle assembly. This method may optionally include, prior to the characterization, isolating, concentrating or purifying the nanoparticulate assembly from the aqueous solution, wherein the native structure of the membrane protein is retained in the isolated nanoparticulate assembly.

Characterization may be either a structural characterization or a functional characterization of the membrane protein, or both. Suitable membrane protein characterization methods include solution and solid state nuclear magnetic resonance (NMR), circular dichroism (CD), electron paramagnetic resonance (EPR), Fourier transform infrared spectroscopy (FTIR), resonance Raman spectroscopy, ultraviolet-visible spectroscopy (UV/vis), cryo-electron microscopy (cryo-EM), surface plasmon Raman spectroscopy, sum frequency generation (SFG), fluorescence, including single-molecule fluorescence and coherent anti-Stokes Raman (CARS), small angle x-ray scattering (SAXS), scanning electron microscopy (SEM), atomic force microscopy (AFM) and enzymatic assays membrane protein structure and dynamics can be characterized using NMR techniques. For example, membrane protein-embedded nanodiscs having a diameter of about 20 nm or less can be characterized using solution NMR and protein-embedded nanodiscs having a diameter greater than about 20 nm can be characterized using solid state NMR. Advantageously, the nanodiscs of the disclosure can include additional features for enhancing characterization by NMR, for example, the nanodisc can be characterized in that when a magnetic field is applied, the nanodisc aligns with the magnetic field and the nanodisc optionally includes a chelating group having a metal ion bound thereto which allows paramagnetic resonance characterization.

In some embodiments, characterizing comprises characterization of the native structure of a membrane protein embedded in a NCMN assembly, the characterization comprising performing at least one of solution nuclear magnetic resonance (NMR), solid state NMR, circular dichroism, electron paramagnetic resonance (EPR), Fourier transform infrared spectroscopy (FTIR), resonance Raman spectroscopy, ultraviolet-visible spectroscopy (UV/vis), cryo-electron microscopy (cryo-EM), surface plasmon Raman spectroscopy, sum frequency generation (SFG), fluorescence, small angle x-ray scattering (SAXS), scanning electron microscopy (SEM), atomic force microscopy (AFM), and an enzymatic assay. In one embodiment, the native structure is characterized by NMR. In another embodiment, the native structure is characterized by cryo-EM.

In some other embodiments, characterizing comprises characterization of the structure of the protein in a preteoliposome. The term “proteoliposome”, as used herein, refers to a system that mimics lipid membranes or liposomes to which a protein has been incorporated or inserted. The proteoliposome (PL) may represent a liposome, a vesicle or any platform that is reconstituted to preserve the embedded protein's (i.e., membrane protein) structural and functional integrities.

The polymers described herein can extract membrane proteins from cell membrane for functional study and high-resolution structure determination. The methods and systems may be used in the drug discovery field using membrane proteins as drug targets. In addition, the methods and systems described herein are Cryo-EM compatible. The methods and systems further allow to extract and purify membrane proteins in their functional forms, thus allowing reproduction and/or further analysis of membrane proteins' activity, including catalytic activity, channel transport activity, protein interaction activity, etc. As part of the NCMN system, a functionalized polymer library has been generated and other various derivatives of functionalized polymers may also be included in the system.

### EXAMPLES

### Example 1: Synthesis of Amide-Grafted NCMN Polymers with Different Grafting Degrees

Poly(styrene-co-maleic anhydride) with a 2:1 ratio of styrene to MAnh (SMAnh, acid number: 355 mg KOH·g⁻¹) was purchased from Cray Valley. (6.0 g, 38.04 mmol, 1 equiv.) was dissolved in 5 mL H₂O. Taurine (0.714 g, 5.7 mmol., 15% equivalent for making 15% grafting; 1.19 g, 9.51 mol, 25% equivalent for making 25% grafting; 2.38 g, 19.02 mmol, 50% equivalent for making 50% grafting, respectively) was dissolved in 5 mL H₂O and its pH was adjusted to 7. The taurine solution was then added into the SMAnh solution and the pH was again adjusted to 7. 1-Ethy-3-(3-dimethylaminopropyl)carbodiimide (EDC)/HCl (1.312 g, 6.84 mmol, 0.3 equivalent for making 15% grafting; 2.187 g, 11.41 mmol, 0.3 equivalent for making 25% grafting; 4.374 g, 22.82 mol, 0.6 equivalent for making 50% grafting, respectively) was then added. The reaction mixture was kept stirring at room temperature for 6 hours and was then neutralized by NaOH 5M and the polymer product were precipitated in ethanol. It was found the amide bonding was formed in each product (NCMN Polymer1-15(AM), NCMN Polymer1-25(AM), NCMN Polymer1-50(AM)).

### Example 2: Characterization and Activity Assay of Listeria monocytogenes Ca²⁺-ATPase (LMCA1)

### Materials and Methods

The LMCA1 plasmid (pET22b-LMCA1) was transformed into E. coli C43 (DE3) competent cells. A single colony was inoculated in 25 ml terrific broth (TB) media supplemented with ampicillin 100 μg/mL and shaken overnight at 37° C., at 250 rpm. 4 ml of the overnight grown cells were diluted in fresh 1 liter TB media containing ampicillin and grew at 37° C. until the O.D.₆₀₀ reached 0.8-1.0. The culture was then cooled to 20° C., induced the recombinant protein expression with 1 mM IPTG (Isopropyl β-D-1 thiogalactopyranoside), and shaken at 20° C. for 22 h. The cells were harvested using a centrifuge force at 7,000×g at 4° C. for 10 min. Cell pellets were stored at −80° C. for further purification.

Ca²⁺-ATPases are membrane pumps that transport calcium ions across the cell membrane and are dependent on ATP. Listeria monocytogenes Ca²⁺-ATPase (LMCA1) was characterized using the detergent-free Native Cell Membrane Nanoparticles (NCMN) system.

### Preparation of LMCA1

The NCMN-LMCA1 particles were prepared according to a method previously reported with modifications. See K. G. Kroeck, et. al., Journal of Visualized Experiments (161) (2020) e61298. The cell pellets were resuspended in NCMN Buffer A (50 mM HEPES, pH 8.4, 500 mM NaCl, 5% glycerol, 20 mM imidazole, 0.1 mM TCEP), and the cells were broken by a high-pressure homogenizer (Avestin EmulsiFlex-C3). Cell debris was removed by centrifugation with 15,000×g at 4° C. for 30 min. Then cell membranes were collected by ultracentrifuge at 299,602×g for 60 min at 4° C. Two grams of overexpressed LMCA1 membrane fraction were suspended in NCMN Buffer A and then homogenized three times with a glass Dounce homogenizer. The membrane-active polymer (NCMN Polymer1-25(AM)) was added to the homogenized membrane fraction at a final concentration of 1.25% w/v in 100 ml. The sample was shaken overnight at room temperature on a nutating shaker. The insoluble pellets were extracted using a 208,057×g centrifuge at 20° C. for 60 min. The supernatant containing membrane proteins was collected and loaded onto a prepacked 5 ml Ni-NTA column (GE Health), pre-equilibrated with NCMN buffer A. The column was washed using stepwise procedure using NCMN buffer B (25 mM HEPES, pH7.8, 500 mM NaCl, 40 mM imidazole, 0.1 mM TCEP, 0.05% NCMN Polymer1-25(AM)), buffer C (25 mM HEPES, pH 7.8, 500 mM NaCl, 75 mM imidazole, 0.1 mM TCEP, 0.05% NCMN Polymer1-25(AM). The LMCA1 protein was eluted with NCMN buffer H (25 mM HEPES, pH 7.8, 200 mM NaCl, 250 mM imidazole, 0.1 mM TCEP, 0.05% NCMN Polymer1-25(AM)), respectively. The purity of the purified protein was evaluated on an SDS-PAGE gel.

The LMCA1 was purified using dodecylmaltoside (DDM) according to the above-mentioned protocol with some modifications. Two grams of homogenized membrane fraction was solubilized with 1% DDM for 1 hr at 4° C. The supernatant containing membrane proteins after 208,057×g centrifuge was collected and loaded onto a prepacked 5 ml Ni-NTA column (GE Health), pre-equilibrated with buffer A (50 mM HEPES, pH7.8, 300 mM NaCl, 5% glycerol, 20 mM imidazole, 1 mM MgCl₂, 0.1 mM TCEP). The column was washed using stepwise procedure using buffer B (50 mM HEPES, pH7.8, 300 mM NaCl, 5% glycerol, 40 mM imidazole, 5 mM MgCl₂, 0.1 mM TCEP, 0.05% DDM), buffer C (25 mM HEPES, pH 7.8, 500 mM NaCl, 5% glycerol, 75 mM imidazole, 0.1 mM TCEP, 0.05% DDM. The LMCA1 protein was eluted with DDM buffer H (25 mM HEPES, pH 7.8, 200 mM NaCl, 5% glycerol, 250 mM imidazole, 0.1 mM TCEP, 0.05% DDM), respectively. Furthermore, size exclusion chromatography was performed using Superdex 200 increase 10/300 pg (GE Healthcare), which was equilibrated with buffer E (40 mM HEPES, pH7.8, 200 mM NaCl, 0.1 mM TCEP, 0.05% DDM. The purity of the purified protein was evaluated on an SDS-PAGE gel.

### ATPase Assay for LMCA1

The ATPase activity was performed following the reported procedure using a malachite green phosphate test kit (Catalog: mak307, Sigma-Aldrich) under its guideline. See C. S. Rule, et. al., Journal of Visualized Experiments, (114) (2016) e54305. A reaction contained DDM or NCMN Polymer1-25(AM) purified LMCA1 protein with reaction buffer of four different pH buffers (50 mM CAPS pH 10.0, 150 mM KCl, 2 mM MgCl₂, 1 mM EGTA), (50 mM Tris pH 9.0, 150 mM KCl, 2 mM MgCl₂, 1 mM EGTA), (50 mM Tris pH 7.5, 150 mM KCl, 2 mM MgCl₂, 1 mM EGTA), (50 mM Bis-Tris pH 6.0, 150 mM KCl, 2 mM MgCl₂, 1 mM EGTA) in the presence and absence of 1.3 mM CaCl₂. The protein-containing buffer solutions were preincubated at 37° C. for 5 mins before activation with an ATP-Mg mixture (with a final concentration of 1 mM). They were then incubated at 37° C. for 30 mins. Finally, 20 μL of reagent solution from the kit was added. The color intensity was recorded at 620 nm using BioTek CYTATION 5 imaging reader. The effect of EGTA (10 mM), and vanadate (1 or 10 mM), at pH 7.5 or 9.0 was studied with NCMN Polymer1-25(AM) purified LMCA1. Each sample would diminish the negative control value without protein as a background. The final absorbance readings were turned into free inorganic phosphate measurements with standard phosphate. ATPase activity was measured as Pi nmol produced by mg protein per minute of reaction time, as determined by three independent tests.

The eluted fraction of LMCA1 with NCMN Polymer1-25 was analyzed and showed a higher purity of NCMN Polymer1-25 purified LMCA1 than the LMCA1 purity after a two-step purification procedure conducted with DDM. A molecular weight of around 95 kDa was observed on SDS-PAGE gel for purified LMCA1, which corresponds to the estimated value.

Using a malachite green phosphate assay kit (Sigma), the ATPase activity of LMCA1 was determined at four distinct pH conditions: pH 6.0, pH 7.5, pH 9.0, and pH 10.0. The results showed that, with calcium ions present, LMCA1 purified with DDM has a maximum ATPase activity of about 160 nmol Pi/min/mg protein at pH 9.0. In contrast, NCMN Polymer1-25 purified LMCA1 had a maximum ATPase activity of about 300 nmol Pi/min/mg protein at pH 9.0. In addition, the NCMN Polymer1-25 purified LMCA1 also showed significant higher ATPase activities at pH 7.5 and 10 than DDM purified LMCA1.

### Example 3: Synthesis of NCMN Polymers with Two Different Grafted Branches

SMAnh polymer (7.5 g, 47.54 mmol COOH, 1 equiv.) was dissolved in 35 mL H₂O and its pH value was adjusted to 6. Taurine (1.487 g, 11.885 mmol, 0.25 equiv.) was dissolved in 15 mL H₂O and its pH was adjusted to 6. The taurine solution was then added into the above polymer solution and the pH was adjusted to 6 again. EDC/HCl (2.734 g, 14.262 mmol, 0.3 equiv.) was then added and the reaction mixture was stirred at room temperature for 4 hours, at the end of which period EDC/HCl (2.734 g, 14.262 mmol, 0.3 equiv.) was added and then Tris (1.439 g, 11.885 mmol, 0.25 equiv.) was added. The reaction mixture was kept stirring at room temperature for 24 hours and was then neutralized by NaOH 5M. NCMN Polymer2-50(AM), which was 50% amide-grafted with taurine and Tris at the ratio of 1:1, was precipitated in cold ethanol.

NCMN Polymer2-50(AM) obtained above was heated at 180° C. in a vacuum oven for 2 hours, during which the amide group in the reactant was reacted to the adjacent carboxylic acid groups to form an imide group. The resulting imide-grafted products are NCMN Polymer2-100(IM).

NCMN Polymer2-50(AM) and NCMN Polymer2-100(IM) were both active to solubilize MP into discoidal particles highly stable toward Ca²⁺ and pH and capable of performing the biological function (see Table 1).

TABLE 1
Structure composition of NMN Polymers and the stability of AcrB
particles after membrane solubilization.

| | Taurine:Tris/ | Imidation | Particle | | Tolerance |
|---|---|---|---|---|---|
| | —COOH | degree | size | Optimum | to [Ca²⁺] |
| Polymer | ratio^{a} | (%)^{b} | (nm)^{c} | pH^{d} | (mM)^{d} |
| SMA2000 | 0 | 0 | ~10 | >5.5 | <1.25 |
| NCMN | 0.25:0.25 | 50 | Mixture | >1 | ≤50.0 |
| Polymer2- | | | | | |
| 50(AM) | | | | | |
| NCMN | 0.25:0.25 | 100 | ~10 | >1 | ≤25.0 |
| Polymer2- | | | | | |
| 100(IM) | | | | | |

^{a}Molar ratio.
^{b}Estimated imidation percentage determined based on ¹H NMR and FTIR characterization.
^{c}TEM characterization.
^{d}Stability of AcrB particles in various buffer environments determined based on turbidity experiments.

### Example 4: Synthesis of Imide-Grafted NCMN Polymers

NCMN Polymer1-15(AM), NCMN Polymer1-25(AM), and NCMN Polymer1-50(AM) obtained in Example 1 were each heated at 180-200° C. in a vacuum oven for 2 hours, during which the amide groups in the reactants were reacted to the adjacent carboxylic acid groups to form imide groups. The resulting imide-grafted products are NCMN Polymer1-30(IM), NCMN Polymer1-50(IM), NCMN Polymer1-100(IM).

NCMN Polymer1-30(IM), NCMN Polymer1-50(IM), NCMN Polymer1-100(IM) were active to solubilize different MPs (e.g., AcrB, MscS, YnaI, MsbA, Lmcr1) into discoidal particles highly stable toward Ca² and pH and capable of performing the biological function (Table 2). Analyzing composition of the encased lipid bilayer confirmed that no particular lipid was enriched during the extraction process. It was observed that the sulfonating degree and conformations influenced the solubilization efficacy, particle size and sensitivity of the resulting particles. Notable success of these sulfonated polymers is providing stable nanodiscs with large quantities and high purity that are applicable for structural studies by cryo-EM and X-ray crystallography. Finally, unexpected fluorescent emission was found in this polymeric series that is adequate for protein labeling and other fluorescent applications without fluorescent tag assistance.

TABLE 2
Structure composition of NMN polymers and the stability of AcrB
particles after membrane solubilization.

| | Taurine/ | Imidation | Particle | | Tolerance |
|---|---|---|---|---|---|
| | —COOH | degree | size | Optimum | to [Ca²⁺] |
| Polymer | ratio^{a} | (%)^{b} | (nm)^{c} | pH^{d} | (mM)^{d} |
| SMA2000 | 0 | 0 | ~10 | >5.5 | <1.25 |
| NCMN | 0.15 | 30 | ~10 | >1 | ≤15.0 |
| Polymer1- | | | | | |
| 30(IM) | | | | | |
| NCMN | 0.25 | 50 | ~10 | >1 | ≤25.0 |
| Polymer1- | | | | | |
| 50(IM) | | | | | |
| NCMN | 0.50 | 100 | ~10 | >1 | ≤25.0 |
| Polymer1- | | | | | |
| 100(IM) | | | | | |

^{a}Molar ratio.
^{b}Estimated imidation percentage determined based on ¹H NMR and FTIR characterization.
^{c}TEM characterization.
^{d}Stability of AcrB particles in various buffer environments determined based on turbidity experiments.

### Example 5: Synthesis of DIBMA Grafted Polymer

Commercially available DIBMA polymer (4 g, 35.2 mmol COOH, 1 equiv.) was dissolved in 20 mL H₂O and its pH value was adjusted to 6. Taurine (2.25 g, 17.952 mmol, 0.51 equiv.) was dissolved in 15 mL H₂O and its pH was adjusted to 6. It was added into the above DIBMA solution and the pH was adjusted again to 6. EDC/HCl (4.386 g, 22.88 mmol, 0.65 equiv.) was then added and the reaction mixture was stirred at room temperature for 24 hours and was then neutralized with neutralized by NaOH 5M. NCMN Polymer3-50(AM) was precipitated in cold ethanol.

NCMN Polymer3-50(AM) was heated at 180-200° C. in a vacuum oven for 2 hours. The amide groups in the reactant was reacted to the adjacent carboxylic acid groups to form imide groups. The resulting product was NCMN Polymer3-100(IM).

Membrane solubilization testing revealed that NCMN Polymer3-100(IM) was an effective solubilizer toward wider range of membrane proteins (MPs) such as AcrB, MscS, Lmcr1 as compared to DIBMA. It also showed promising performances in the solubilization of different MPs in term of yield, purity and homogeneity. Likewise, the long-term stability and pH- and divalent tolerance of NCMN Polymer3-100(IM) particles are much better than DIBMA and SMA (Table 3).

TABLE 3
Structure composition of NMN polymers and the stability of AcrB
particles after membrane solubilization.

| | Particle | | Tolerance |
|---|---|---|---|
| | size | Optimum | to [Ca²⁺] |
| Polymer | (nm)^{a} | pH^{b} | (mM)^{b} |
| SMA | ~10 | >5.5 | ≤1.25 |
| DIBMA | Various size | >4 | ≤20 |
| NCMN Polymer3-100(IM). | ~10 | All range | ≤50 |

^{a}TEM characterization.
^{b}Stability of AcrB particles in various buffer environments determined based on turbidity experiments.

### Example 6 Synthesis of NCMN Polymer Grafted with Both Anion Branch and Cation Branch

NCMN Polymer4-50(AM), grafted with DMEDA and Taurine at the ratio of 1:1 and the 50% grafting degree, was prepared by first reacting SMA (1 equiv.) with N,N-dimethylethylenediamine (DMEDA, 0.25 equiv.) at 100° C. The resulting reaction mixture was hydrolyzed with NaOH at 110° C., and then reacted with Taurine (0.25 equiv.) in the presence of EDC at room temperature. Membrane solubilization testing showed that this polymer was an effective solubilizer toward membrane protein (MP) with notable improvement in divalent cation-sensitivity (Table 4).

TABLE 4
Structure composition of NMN polymers and the stability of AcrB
particles after membrane solubilization.

| | Taurine: | | | | |
|---|---|---|---|---|---|
| | DMEDA/ | Amidation | Particle | | Tolerance |
| | —COOH | degree | size | Optimum | to [Ca²⁺] |
| Polymer | ratio^{a} | (%)^{b} | (nm)^{c} | pH^{d} | (mM)^{d} |
| SMA2000 | 0 | 0 | ~10 | >5.5 | <1.25 |
| NCMN | 0.25:0.25 | 50 | Mixture | >6 | ≤15.0 |
| Polymer4- | | | | | |
| 50(AM) | | | | | |

^{a}Molar ratio.
^{b}Estimated imidation percentage determined based on ¹H NMR and FTIR characterization.
^{c}TEM characterization.
^{d}Stability of AcrB particles in various buffer environments determined based on turbidity experiments.

It is to be understood that this invention is not limited to any particular embodiment described herein and may vary. It is also to be understood that the terminology used herein is for the purpose of describing particular embodiments only, and is not intended to be limiting, since the scope of the present invention will be limited only by the appended claims.

Where a range of values is provided, it is understood that each intervening value between the upper and lower limit of that range (to a tenth of the unit of the lower limit) is included in the range and encompassed within the invention, unless the context or description clearly dictates otherwise. In addition, smaller ranges between any two values in the range are encompassed, unless the context or description clearly indicates otherwise.

Unless defined otherwise, all technical and scientific terms used herein have the same meaning as commonly understood by one of ordinary skill in the art to which this invention belongs. Representative illustrative methods and materials are herein described; methods and materials similar or equivalent to those described herein can also be used in the practice or testing of the present invention.

All publications and patents cited in this specification are herein incorporated by reference as if each individual publication or patent were specifically and individually indicated to be incorporated by reference, and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual dates of public availability and may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.) . . . ”.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

What is claimed is:

1. A functionalized polymer comprising: (1) a copolymer backbone and (2) at least one functional branch;
wherein the copolymer backbone has a number average molecular weight of 1,000-18,000 Da;
wherein said functional branch is C₁-C₈ straight or branched alkyl substituted with an ionic functional group of 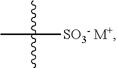
and is attached to an acyl moiety of said copolymer backbone by an amide or ester bonding, or attached to two acyl moieties of the copolymer backbone by an imide bonding; and
wherein said alkyl group optionally contains one or more heteroatoms selected from the group consisting of nitrogen, oxygen and sulfur, and M⁺ is NH₄⁺, Na⁺ or K⁺, or M⁺ is another branch of the functionalized polymer which contains a cation of 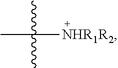
each of R₁ and R₂ independently being H or C₁-C₆ alkyl.

2. The functionalized polymer of claim 1, wherein the backbone is a copolymer radical having a number average molecular weight of 5,000-18,000 Da.

3. The functionalized polymer of claim 2, wherein the functional branch is attached to the acyl moiety of the copolymer backbone by an amide or ester bonding and comprises a moiety of the following: 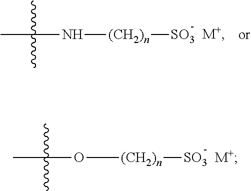
wherein n is 1, 2, 3, 4, 5, or 6; and M⁺ is defined in claim 1.

4. The functionalized polymer of claim 2, wherein the functional branch is attached to the two acyl moieties of the copolymer or peptide backbone by an imide bonding and comprise a moiety of the following: 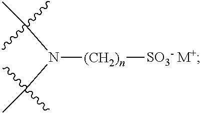
wherein n is 1, 2, 3, 4, 5, or 6; and M⁺ is defined in claim 1.

5. The functionalized polymer of claim 3, wherein the backbone is a copolymer radical derived from SMA, which has a number average molecular weight between 6000 and 10,000 Daltons and a ratio of styrene monomer units to maleic monomer units between 0.5:1 and 10:1; and wherein the functional branch comprises a moiety of the following: 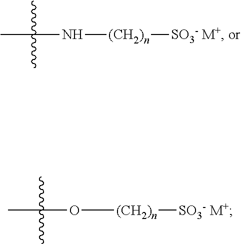
wherein n is 2, 3, or 4; and M⁺ is Na⁺.

6. The functionalized polymer of claim 4, wherein the backbone is a copolymer radical derived from SMA, which has a number average molecular weight between 6000 and 10,000 Daltons and a ratio of styrene monomer units to maleic monomer units between 0.5:1 and 10:1; and wherein the functional branch comprises a moiety of the following: 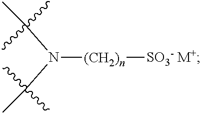
wherein n is 2, 3, or 4; and M⁺ is Na⁺.

7. The functionalized polymer of claim 3, wherein the backbone is a copolymer radical derived from DIBMA, which has a number average molecular weight between 8,000 and 16,000 Daltons and a ratio of diisobutylene monomer units to maleic monomer units between 0.5:1 and 10:1; and wherein the functional branch comprises a moiety of the following: 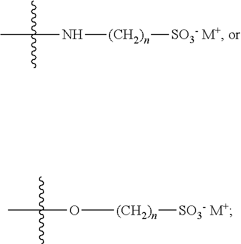
wherein n is 2, 3, or 4; and M⁺ is Na⁺.

8. The functionalized polymer of claim 4, wherein the backbone is a copolymer radical derived from DIBMA, which has a number average molecular weight between 8,000 and 16,000 Daltons and a ratio of diisobutylene monomer units to maleic monomer units between 0.5:1 and 10:1; and wherein the functional branch comprises a moiety of the following: 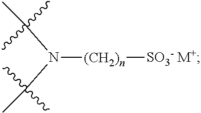
wherein n is 2, 3, or 4; and M⁺ is Na⁺.

9. The functionalized polymer of claim 1, wherein the functionalized polymer has a structure selected from a group consisting of:

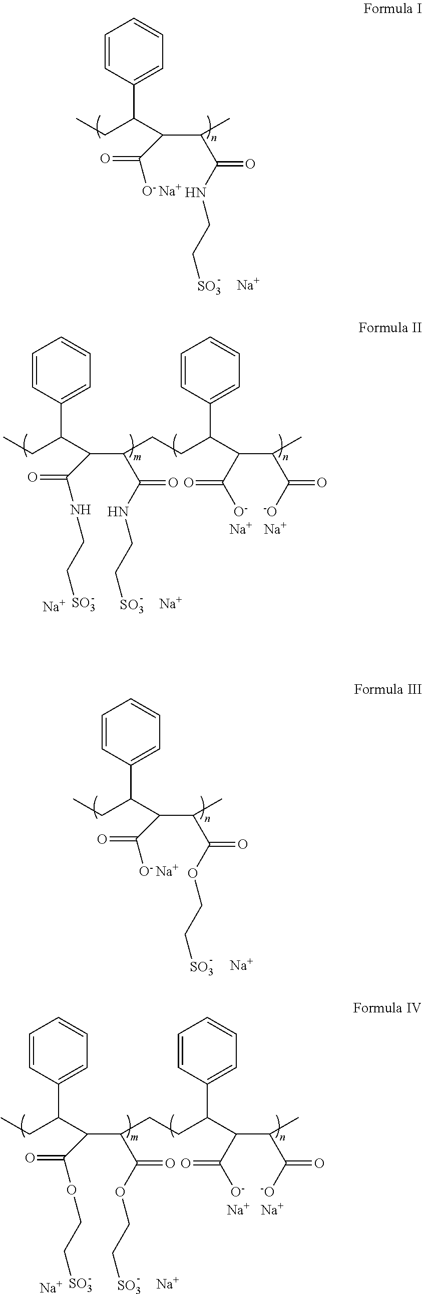

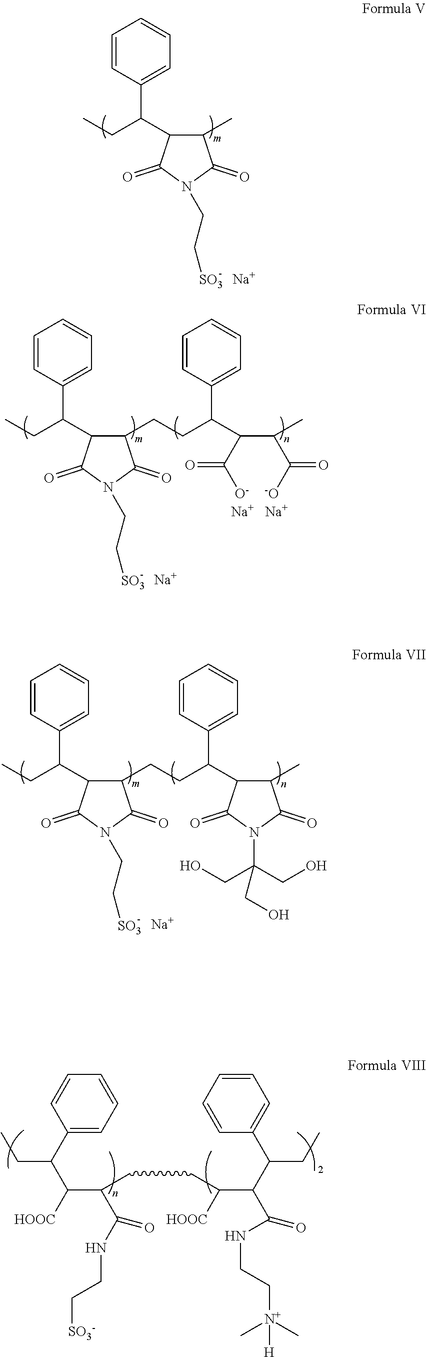

10. The functionalized polymer of claim 1, wherein the functionalized polymer contains more than one functional branches and the grafting degree of the functionalized polymer is 15%-75%.

11. The functionalized polymer of claim 1, wherein the functionalized polymer contains more than one functional branches and the grafting degree of the functionalized polymer is 25%-75%.

12. The functionalized polymer of claim 1, wherein the functionalized polymer contains more than one functional branches and the grafting degree of the functionalized polymer is 15%-50%.

13. The functionalized polymer of claim 1, wherein the functionalized polymer contains more than one functional branches and the grafting degree of the functionalized polymer is 25%-50%.

14. The functionalized polymer of claim 5, wherein the ratio of styrene monomer units and maleic monomer units of the copolymer backbone is between 0.5:1 and 10:1.

15. The functionalized polymer of claim 14, wherein the functionalized polymer contains more than one functional branches and the grafting ratio of the functionalized polymer is 15%-75%.

16. The functionalized polymer of claim 1, wherein the functionalized polymer further comprises one or more second branches, which are different from the at least one functional branch.

17. A native cell membrane nanoparticle system comprising:
(1) one or more functionalized polymers of claim 1, and
(2) a portion of a cell membrane containing at least one membrane protein and membrane lipid associated thereto;
wherein the one or more functionalized polymers encapsulate the membrane protein and the associated membrane lipid to form a nanoparticle assembly and wherein the native structure of the membrane protein is retained in the nanoparticle assembly.

18. The native cell membrane nanoparticle system of claim 17, wherein the nanoparticle assembly is soluble in water.

19. The native cell membrane nanoparticle system of claim 18, wherein the cell membrane protein is selected from a group consisting of integral membrane proteins, peripheral and monotopic membrane proteins, G protein receptors, and Type I, II and III cell-surface receptors.

20. The native cell membrane nanoparticle system of claim 18, wherein the system is substantially free of a detergent.

21. A method of extracting a membrane protein comprising:
(1) contacting a portion of a cell membrane containing at least one membrane protein and lipid associated thereto with a functionalized copolymer of claim 1 in an aqueous solution to form a nanoparticle assembly, in which the portion of the cell membrane is encapsulated by the functionalized copolymer, and
(2) isolating the nanoparticulate assembly from the aqueous solution;
wherein the native structure of the membrane protein is retained in the isolated nanoparticulate assembly.

22. A method of characterizing the native structure of a membrane protein, comprising:
(1) contacting a portion of a cell membrane containing at least a membrane protein and lipid associated thereto with a functionalized copolymer of claim 1 in an aqueous solution to form a nanoparticle assembly, in which the portion of the cell membrane is encapsulated by the functionalized copolymer,
(2) isolating the nanoparticulate assembly from the aqueous solution, wherein the native structure of the membrane protein is retained in the isolated nanoparticulate assembly, and
(3) subjecting the isolated nanoparticle assembly to cryo-EM or NMR to characterize the native structure of the membrane protein.
